(19) United States
(12) Patent Application Publication
(10) Pub. No.: US 2023/0007922 A1
(43) Pub. Date: Jan. 12, 2023

(54) Method and System for Estimating Physiological Parameters Utilizing a Deep Neural Network to Build a Calibrated Parameter Model

(71) Applicant: ChroniSense Medical Ltd., Yokneam (IL)

(72) Inventor: Daniel H. Lange, Kfar Vradim (IL)

(21) Appl. No.: 17/900,793

(22) Filed: Aug. 31, 2022

Related U.S. Application Data
(63) Continuation-in-part of application No. 15/854,628, filed on Dec. 26, 2017, now Pat. No. 11,464,457.
Application No. 15/854,628 is a continuation-in-part of application No. 14/738,636, filed on Jun. 12, 2015.
Application No. 14/738,636 is a continuation-in-part of application No. 17/532,966, filed on Nov. 22, 2021.

(51) Int. Cl. A61B 5/00; A61B 5/021

(52) U.S. Cl. CPC A61B 5/7267; A61B 5/021; A61B 5/7275; A61B 2560/0223

(57) ABSTRACT

A method and system are provided for estimating a physiological parameter using a parameter model determined by a deep neural network. An example method includes training a deep neural network with indirect and direct physiological parameters from a user database. The medical parameters include a respiratory rate, oxygen saturation, temperature, blood pressure, and pulse rate. The method includes determining if a new user belongs in a group. If the parameter model estimated physiological parameter using the closest group to the new user and associated calibration, then the method quantizes the parameter inputs to determine which physiological parameter a new user is most sensitive and to determine a new group and calibration coefficients or curves for the new user.

### CROSS-REFERENCE TO RELATED APPLICATIONS

The present application is a Continuation-in-Part of U.S. patent application Ser. No. 15/854,628, titled “Determining an Early Warning Score Based on Wearable Device Measurements”, filed on Dec. 26, 2017, and a Continuation-in-Part of U.S. patent application Ser. No. 14/738,636, titled “Wearable Device Electrocardiogram”, filed on Jun. 12, 2015. The present application is also a Continuation-in-Part of U.S. patent application Ser. No. 17/532,966, titled “Error Correction in Measurement of Medical Parameters” filed Nov. 22, 2021. The present application is related to U.S. patent application Ser. No. 14/738,666, titled “Monitoring Health Status of People Suffering from Chronic Diseases”, filed on Jun. 12, 2015, now U.S. Pat. No. 11,160,459 issued on Nov. 2, 2021. The present application is also related to U.S. patent application Ser. No. 14/738,711, titled “System for Performing Pulse Oximetry”, filed on Jun. 12, 2015, now U.S. Pat. No. 10,470,692 issued on Nov. 12, 2019. The disclosures of the aforementioned applications are incorporated herein by reference for all purposes, including all references cited therein.

### FIELD

The present application relates to systems and methods for monitoring health status of people, and more specifically systems and method for determining an early warning score (EWS) based on wearable device measurements. Further, the application relates to the improved estimation of physiological parameters using deep neural networks to develop a parameter model and associated calibration coefficients.

### BACKGROUND

It should not be assumed that any of the approaches described in this section qualify as prior art merely by virtue of their inclusion in this section.

Monitoring health status of a patient and progression of chronic diseases, which includes measuring medical parameters, is central for providing appropriate and timely treatment to patients suffering from such chronic diseases as chronic heart failure, cardiac arrhythmia, chronic obstructive pulmonary disease, asthma, and diabetes. Recently, an EWS technique was introduced to facilitate estimation of a degree of illness of a patient. The EWS can be determined based on medical parameters, such as a respiratory rate, oxygen saturation, temperature, blood pressure, pulse rate, and level consciousness. Traditionally, monitoring is carried out and measurements are taken while a patient is hospitalized or in other clinical settings. Appropriate treatment regimen can be based on these measurements, and thus it is highly beneficial to monitor medical parameters of the patient after the patient is released from the hospital. Therefore, the patient can be asked to visit the hospital or clinic periodically for monitoring, and adjustment of treatment, if necessary. However, most often, no measurements are carried out between visits, usually due to the need for trained examiners and medical devices. This is unfortunate, because between visits the chronic disease from which the patient suffers can worsen and result in emergency treatment and hospitalization. Furthermore, after receiving repeated courses of emergency hospital treatment, the patient's health condition may degrade and never return to the pre-hospitalization level. Therefore, a technology that allows for at-home measurements can be essential to managing chronic diseases or even saving a patient's life. Early warnings of worsening conditions associated with chronic diseases may prevent unnecessary hospitalizations by providing a preventive treatment and, as a result, reduce financial and human costs of the hospitalization and treatment.

Currently there are no user-friendly devices for continuous non-intrusive measurements of medical parameters of patients at their home or working environment. In some cases, patients with severe symptoms can be monitored at home while staying in bed. However, devices for taking measurements of bedridden patients are generally not suitable for chronic patients which, with timely treatment, should be able to maintain a high-quality normal life.

Some existing mobile devices can provide functionalities for tracking people's physical activity. Such devices can measure a pulse rate and the distance a person walks or runs, calculate burned calories, and so forth. Some of these existing devices are designed as or are part of a watch, a bracelet, and a wristband. However, these devices are primarily designed for healthy people for monitoring of their physical exercise and not for monitoring health status of people.

Another challenge for monitoring people's health status is that monitoring from a watch, a bracelet, and a wristband is often performed utilizing indirect physiological parameter measurements or derived parameter measurements. Using light reflections to measure oxygen levels, is an example of an indirect measurement, using the pulse and heart electrical signals to calculate an estimation of pulse width velocity is an indirect calculation. Blood pressure measurements with an arm cuff are a direct measurement and more accurate than indirect measurements. These indirect measurements can be off by a relevant amount that might indicate that a person's health status is different from what is indicated.

The wearable device measurements, indirect, direct, or determined parameters, are often uploaded to a user account providing a history of the parameter measurements. Additionally, other information can be input by a user when setting up and maintaining an account associated with the wearable device. This information along with potentially millions of other accounts with similar information lend themselves to machine learning with deep neural networks to estimate physiological parameters and identify groups of people with similar bias between the indirect and determined physiological parameter measurements and direct physiological parameter measurements. The DNN can be used in determining a parameter model and calibration coefficient or calibration curve can be determined and applied to the parameters or the output of a parameter model to improve the physiological parameter estimation accuracy. Further, this improved physiological parameter estimate can be used in the determining a health score.

Another challenge is for an AI (artificial intelligence) based medical systems is governmental agencies that approve and/or fund medical devices can require that errors be explainable. When a system utilizing AI makes a mistake, the governmental agency can require an explanation of why the system made the decision it made. This requires that when a new person is added to the system, and if the corrected prediction model's physiological parameter measurement error is too large, a new group needs to be formed with new correction factors.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

According to one embodiment of the present disclosure, a method for estimating a physiological parameter utilizing a parameter model determined by a DNN (deep neural network) is provided. The method accesses a user database containing thousands of users where indirect physiological parameter measurements are uploaded for training the DNN. In one aspect of the invention the indirect physiological parameters are uploaded from a wearable device to the database. These physiological parameters can include respiratory rate, an oxygen saturation, a temperature, a derived blood pressure, and a pulse rate. The direct physiological parameter can be blood pressure measured by an arm cuff which is more accurate than the indirect means.

The database is accessed to utilize the indirect and direct physiological measurements to train a DNN with data from the plurality of users in the database. The DNN may also access and use user attribute information to improve the estimates of a physiological parameter such as blood pressure.

From the user database the users are grouped using the user's physiological parameters. In some embodiments, the DNN determines the groups based on the physiological parameters. Once the groups are determined, then each user in the database is associated with a group. In another embodiment, the groups are determined by a distance determined between the physiological parameters.

The purpose of the groups is to determine group calibration coefficient(s) or curves for each group and use the coefficients and parameter model to generate accurate physiological parameter estimates.

As new people are added to the system, a determination is provided of what group the person is closest to and the predetermined coefficients to be used in providing an improved estimate of a physiological parameter. In one aspect of the invention the physiological parameter is blood pressure.

A parameter model is generated using the trained DNN to derive the parameters. The reason not to directly use the DNN is that a parameter model is more explainable when a new user physiological parameter estimation error does not fit with a group to which the new user is closest. A person skilled in data science modeling would be able to develop a parameter model using the DNN output.

The system next processes new users to generate estimated calibrated physiological parameters. A new user is evaluated to determine its distance from each of the one or more groups that have been determined from the database or by the DNN. The new user's candidate group is the group is selected with the least distance, either by distance of the physiological parameters or error from the parameter model. If the calibrated error is less than a threshold, then the candidate group is associated with the new user and can be stored in the database.

If the error or distance of the new user from the candidate group is greater than a threshold, then the following steps are taken to determine of which one or one or more of the physiological parameters is contributing the most to the error or distance.

The determination of which one or more of the physiological parameters for the new user is performed by incrementally iterating through each of the physiological parameters and quantizing the physiological input to determine if the error between the parameter model and the direct physical parameter measurement increases or decreases. The physiological parameter having the most effect on the error is identified.

A new group is defined based on the new user physiological parameters and stored in the database along with determined associated correction factors or coefficients.

### BRIEF DESCRIPTION OF THE DRAWINGS

Embodiments are illustrated by way of example and not limitation in the figures of the accompanying drawings, in which like references indicate similar elements and in which:

FIG. 1 is a block diagram showing an example system for providing EWS for a health status of a patient.

FIG. 2 is a block diagram showing components of an example device for providing EWS for a health status of a patient.

FIG. 3 is a block diagram illustrating example sensors, example medical parameters, and example chronic diseases.

FIGS. 4A and 4B are schematic diagrams illustrating an example device for providing EWS for a health status of a patient.

FIG. 4C is a block diagram showing an example optical sensor.

FIG. 5 is a block diagram showing an example system for providing EWS for a health status of a patient.

FIG. 6 is a flow chart showing steps of an example method for providing EWS for a health status of a patient.

FIG. 7 is a flow chart showing the steps of training a deep neural network to correct physiological measurement.

FIG. 8 is a flow chart showing the steps of determining and if a new use fits with a group of users.

FIG. 9 is a method for determining new parameter correction factors for a new group.

FIG. 10 is a block diagram of a system for estimating a physiological parameter using a parameter model configured from a DNN.

### DETAILED DESCRIPTION

The following detailed description includes references to the accompanying drawings, which form a part of the detailed description. The drawings show illustrations in accordance with exemplary embodiments. These exemplary embodiments, which are also referred to herein as “examples,” are described in enough detail to enable those skilled in the art to practice the present subject matter. The embodiments can be combined, other embodiments can be utilized, or structural, logical and electrical changes can be made without departing from the scope of what is claimed. The following detailed description is, therefore, not to be taken in a limiting sense, and the scope is defined by the appended claims and their equivalents.

The present disclosure provides systems and methods for providing EWS of a health status of a patient. The patient may suffer from chronic diseases. Embodiments of the present disclosure can allow measuring medical parameters of a patient in a non-intrusive manner while, for example, the patient is at home, at work, outdoors, traveling, and at other stationary or mobile environments. Some example embodiments can provide for a wearable device (e.g., a wristband, a watch, or a bracelet) that includes sensors configured to measure medical parameters such as, for example, blood pressure, heart rate, blood oxygen saturation, respiration, and the like. The measurements can be taken during daytime and nighttime for days, weeks, months, and years. The medical parameters can be analyzed to determine trends in the medical parameters and an EWS for health status of the patient. The EWS can be further used to determine whether the severity of the patient's chronic disease (e.g., a heart disease, diabetes, lung disease, and so on) worsens or improves. Embodiments of the present technology may facilitate a rapid reaction to provide an appropriate and timely treatment for the patient. The early treatment may allow taking timely measures to avoid worsening of the patient's condition to the point of requiring an emergency hospitalization and associated expensive medical treatment.

According to various example embodiments, a method for providing the EWS of a health status of a patient includes acquiring, during a predetermined time period, by at least one processor communicatively coupled to sensors integrated into a wearable device, initial values of medical parameters of a patient. The wearable device can be designed to be worn on a wrist of the patient. The method may include determining, by the at least one processor and based on the initial values, normal values of the medical parameters. The method may further include acquiring, by the at least one processor via the sensors and at a pre-determined frequency, further values of the medical parameters. The method may further allow determining, by the at least one processor and based on deviations of the further values from the normal values, individual scores for the medical parameters. The method may further include calculating, by the at least one processor and based on the individual scores, a general score. The method may further include determining, by the at least one processor and based at least on the general score, the health status of the patient.

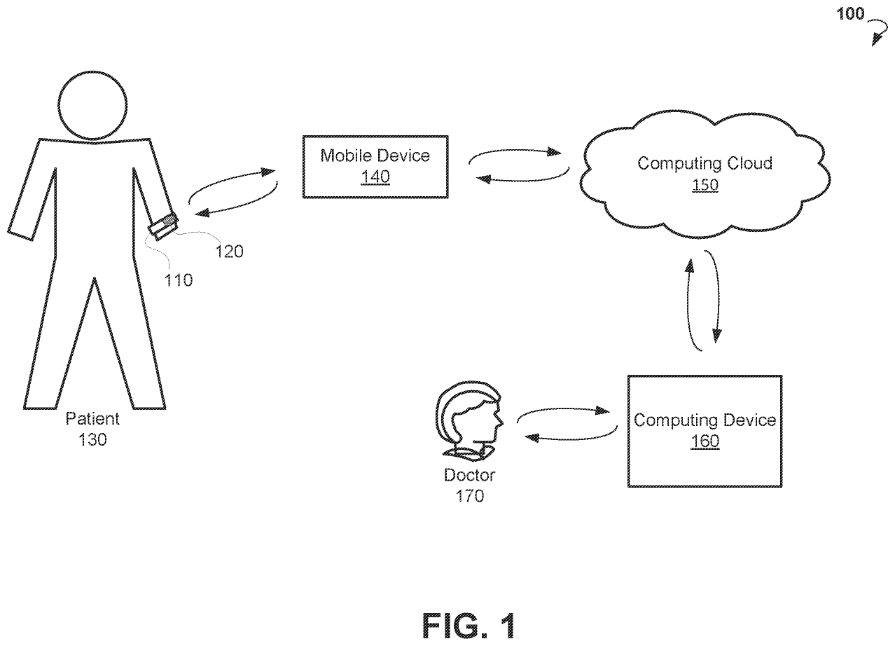

Referring now to FIG. 1, an example system 100 for providing the EWS of a health status of a patient is shown. The system 100 includes at least a wearable device 110. The wearable device includes sensors 120. In some embodiments, the wearable device 110 is worn by a patient 130, for example on a wrist, for an extended period of time. The mobile device can be carried out as a watch, a bracelet, a wristband, and the like.

The wearable device 110 is operable to constantly collect, via sensors 120, sensor data from a patient 130. In some embodiments, based on the sensor data, the wearable device 110 is operable to obtain medical parameters associated with the patient 130. The medical parameters can be analyzed to obtain changes (trends) in medical parameters and the EWS of health status of the patient over time. Based on the changes and the EWS, one or more conclusions regarding severity of one or more chronic disease can be obtained. The wearable device 110 is operable to send messages regarding a current health status to the patient, a relative, a caretaker of the patient, or a doctor treating the patient. The patient 130 can be advised to see a doctor and/or take medicine. In some embodiments, the wearable device 110 analyzes the medical parameters to determine whether the patient has taken the medicine and to provide further advice to the patient.

In various embodiments, the system 100 may further include a mobile device 140. The mobile device 140 can be communicatively coupled to the wearable device 110. In various embodiments, the mobile device 140 is operable to communicate with the wearable device 110 via a wireless connection. The mobile device 140 can include a mobile phone, a smart phone, a phablet, a tablet computer, a notebook, and so forth. The mobile device 140 can be operable to receive the sensor data and medical parameters from the wearable device 110. In certain embodiments, the mobile device 140 is operable to perform analysis of the received sensor data and medical parameters to determine an EWS concerning the health status of the patient. The mobile device 140 can be further configured to provide, based at least on the EWS, a report regarding current health status to the patient 130. In various embodiments, the mobile device 140 runs one or more applications that provide, via a graphical display system, charts and graphics concerning medical parameters of the patient.

In some embodiments, the mobile device 140 is operable to determine the severity of a health status resulting from the chronic disease from which the patient suffers and provide the patient with advice to see a medical professional or to take medicine. An alert message regarding health status of the patient can be sent to a doctor, a relative, or caretaker of the patient.

In further embodiments, the system 100 may further include a cloud-based computing resource 150 (also referred to as a computing cloud). In some embodiments, the cloud-based computing resource 150 includes one or more server farms/clusters comprising a collection of computer servers and is co-located with network switches and/or routers. In certain embodiments, the mobile device 140 is communicatively coupled to the computing cloud 150. The mobile device 140 can be operable to send the sensor data and medical parameters to the computing cloud 150 for further analysis. The computing cloud 150 is operable to store historical data concerning patient health status including sensor data, medical parameters, and EWS collected over days, weeks, months, and years. The computing cloud 150 can be operable to run one or more applications and to provide reports regarding health status of the patient. A doctor 170 treating the patient may access the reports, for example via computing device 160, using the Internet or a secure network. In some embodiments, the results of the analysis of the medical parameters can be sent back to the mobile device 140.

The severity of the health status resulting from a chronic disease can be estimated by computing a deviation or divergence from normal medical parameters of one or more medical parameters being measured at the moment. The normal medical parameters can be specific to the patient 130 and can be derived based on historical data concerning the patient's health status recorded over an extended time period. If the deviation in the medical parameters becomes sufficiently large, the patient can be advised, via a message to the mobile device 140, to take medicine or contact a doctor. In some situations, when the deviation becomes substantial, an alert message can be sent by the mobile device 140 and/or the wearable device 110 to a relative, a doctor, or a caretaker of the patient.

It may be desirable for the patient to be assured that the current medical parameters are within an acceptable deviation of the normal medical parameters. For example, when the current medical parameters are normal, the wearable device 110 and/or mobile device 140 can be operable to periodically alert the patient using a pleasant sound. The signal can be provided, for example, every 30 minutes, once every hour, and the like. In certain embodiments, when the medical parameters are within normal values, the mobile device 140 may provide a text message assuring the patient of normal conditions. A haptic feedback component can be used to alert the patient to a health condition, to warn the patient about a specific event concerning treatment of a chronic disease, to remind the patient to take a medicine, if the patient has failed to take the medicine within a pre-determined period of time, and so forth. The wearable device 110 may include a haptic feedback functionality for providing the patient with haptic feedback, for example, a tap-in device, to apply a force or vibration to skin of the patient. In further embodiments, the haptic alert can be provided by the mobile device 140. The mobile device can vibrate when the mobile device in a pocket of the patient or when the mobile device is located on a surface (e.g., a table).

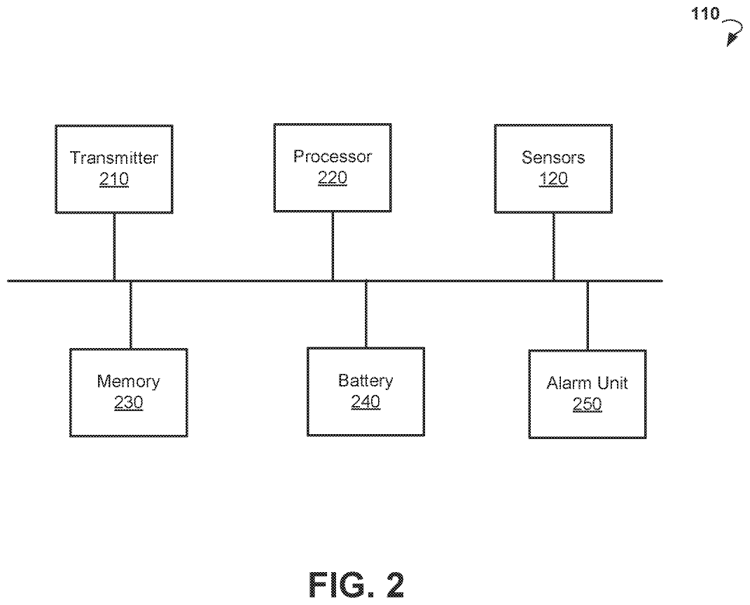

FIG. 2 is a block diagram illustrating components of a wearable device 110, according to an example embodiment. The example wearable device 110 includes sensors 120, a transmitter 210, processor 220, memory storage 230, a battery 240, and an alarm unit 250. The transmitter 210 is configured to communicate with a network such as the Internet, a Wide Area Network (WAN), a Local Area Network (LAN), a cellular network, and so forth, to send a data stream, for example sensor data, medical parameters, and messages concerning the health condition of a patient.

The processor 220 can include hardware and/or software, which is operable to execute computer programs stored in memory 230. The processor 220 can use floating point operations, complex operations, and other operations, including processing and analyzing sensor data to obtain current medical parameters and the EWS of the patient 130.

In some embodiments, the battery 240 is operable to provide electrical power for operation of other components of the wearable device 110. In some embodiments, the battery 240 is a rechargeable battery. In certain embodiments, the battery 240 is recharged using inductive charging technology.

In some embodiments, the alarm unit 250 may include a vibration unit, a tap-in device, a buzzer or a combination of thereof. The alarm unit 250 can be used to provide alarms to patient 130 by creating vibrational tactile sensations in pressure receptors of the skin of the patient or by playing sounds.

The wearable device 110 may comprise additional or different components to provide a particular operation or functionality. In some embodiments, the wearable device 110 may include one or more buttons (or touch screen elements) for entering information. For example, one of the buttons can be used to indicate that the patient is being supplied with external oxygen. In certain embodiments, one of the buttons can be used to enter a level of consciousness of the patient. In other embodiments, the wearable device 210 may include fewer components that perform similar or equivalent functions to those depicted in FIG. 2.

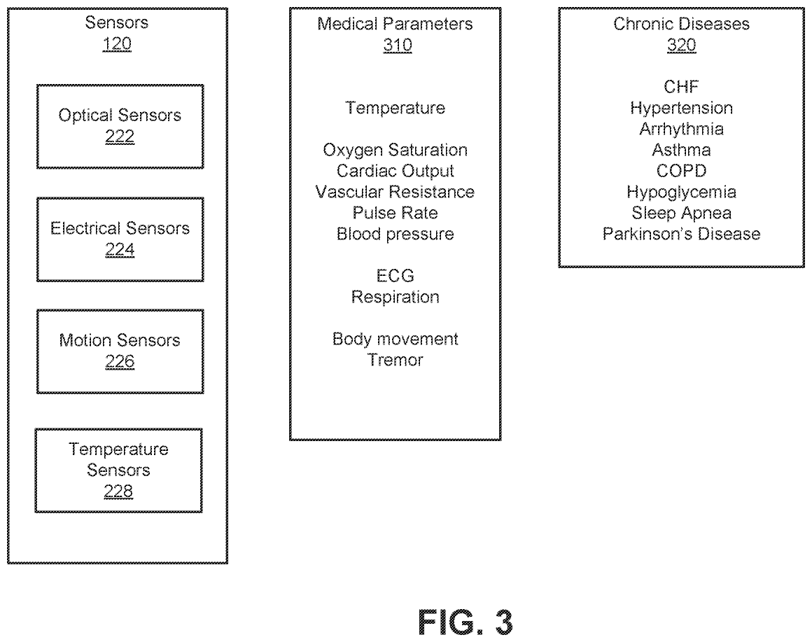

FIG. 3 is a block diagram showing a plurality of example sensors 120, a plurality of example medical parameters 310, and a plurality of example chronic diseases 320. In various embodiments, the sensors 120 include optical sensors 222, electrical sensors 224, motion sensors 226, and temperature sensor 228. The medical parameters 310, determined based on the sensor data, include, but are not limited to, SpO2 oxygen saturation, tissue oxygen saturation, cardiac output, vascular resistance, pulse rate, blood pressure, respiration, electrocardiogram (ECG) data, and motion data. The chronic diseases 320, the progression of which can be tracked based on changes of the medical parameters, include but are not limited to congestive heart failure (CHF), hypertension, arrhythmia, asthma, chronic obstructive pulmonary disease (COPD), hypoglycemia, sleep apnea, and Parkinson's disease.

The optical sensors 222 are operable to measure medical parameters associated with blood flow in an artery (for example, radial artery) using changing absorbance of light at different wavelengths in arterial blood and skin. The optical sensors can determine multiple medical parameters, including but not limited to: SpO2 oxygen saturation, cardiac output, vascular resistance, pulse rate, and respiration. Based on the measurements obtained from optical sensors, abnormal cardiac rhythms (for example, atrial fibrillation, rapid rhythms and slow rhythms) can be detected.

In some embodiments, respiration can be derived from a sinus arrhythmia waveform. The sinus arrhythmia waveform can be obtained based on intervals between subsequent heart beats (RR intervals) measured by the optical sensors using the fact that the rhythm of the heart beats is modulated by human breathing.

The electrical sensors 224 can be operable to obtain electrocardiographic (ECG) activity data of the patient. The ECG activity data includes a characteristic electrically-derived waveform of a heart activity. The ECG data can include a number of components, whose characteristics (timing, amplitude, width, and so forth), alone or in combination, can provide a picture of cardiac and overall health. The ECG data is typically derived by measurements from one or more sets of leads (groups of electrodes comprising grounded circuits), such that the exact characteristics of the resulting waveforms is a function of the electrical and spatial vectors of the electrode positions relative to the heart. While the details of interpretation of the ECG data are too involved to describe succinctly in this disclosure, consideration of each of the component parameters can indicate health status, physical or psychological stress, or trends of disease conditions. Various cardiovascular parameters can be extracted from the ECG alone (such as a heart rate for example), or in combination with other physiological measurements.

ECG-like components can also be obtained, or re-constructed, through other methods of physiological measurements, such as mechano-cardiography, for example. According to example embodiments of present disclosure, ECG of the patient can be measured via the electrical sensors 224. Since measurements are taken from a wrist of the patient, electrodes (or input plates) of the electrical sensors 224 are located very close to each other on a wearable device 110. The electrodes can be positioned to constantly contact the skin of the patient 130 at least two points within a band of the skin surrounded by the wearable device 110. Therefore, the ECG data may contain noise. Averaging of several subsequent intervals of the ECG data between heart beats can be used to cancel out noise in ECG data. To determine intervals between two subsequent heart beats, the pulse wave as measured by optical sensors 222 can be used as a reference. In some embodiments, the pulse rate measured by the optical sensors 222 can be used as a reference signal to improve quality of ECG data of patient when the ECG data are measured from electrodes placed on two limbs (for example two wrists) of the patient. In certain embodiments, an arrhythmia analysis can be carried out using the ECG data and data concerning cardiac output and pulse rate.

In some embodiments, the motion sensors 226 include an accelerometer, gyroscope, and Inertial Measurement Unit (IMU). The motion data obtained via motion sensors 226 can provide parameters of body movement and tremor. The motion data can allow tracking the progression or remission of a motor disease, Parkinson's disease, and physical condition of the patient. In some embodiments, the motion data can be analyzed to determine whether the patient is likely to fall. In some embodiments, the motion data can be analyzed in time domain and frequency domain. By tracking amplitudes of movement of the patient it can be determined if the patient's movements become slower (i.e., the patient becomes sluggish) or the patient is not moving at all.

In some embodiments, the motion data obtained from the motion sensors 226 can be also used to obtain respiration data of the patient. For example, the motion sensors 226 may include a three-dimensional gyroscope. When the patient moves, the three-dimensional gyroscope can measure rotation around axes X, Y, and Z. The signal provided by the gyroscope may include components due to breathing of the patient. In some embodiments, the wearable device 110 can be configured to perform a spectral analysis on the signal provided by the three-dimensional gyroscope to determine a spectrum.

The signal may include rotation around one of the axes X, Y, Z, or a combination of the rotations. The spectrum analysis may include Fourier transform, periodogram-based methods, Bartlett's method, Welch's method, least-squares spectral analyses, and other techniques. The spectrum can be further used to determine a frequency of breathing (respiration). The respiration determined based on motion data may be more reliable than the respiration determined from a sinus arrhythmia waveform, since heart beat intervals measured by optical sensors can be contaminated if the patient suffers from a heart arrhythmia. The respiration measured by the wearable device can be also more reliable than manual counting of breathing carried out by medical personal.

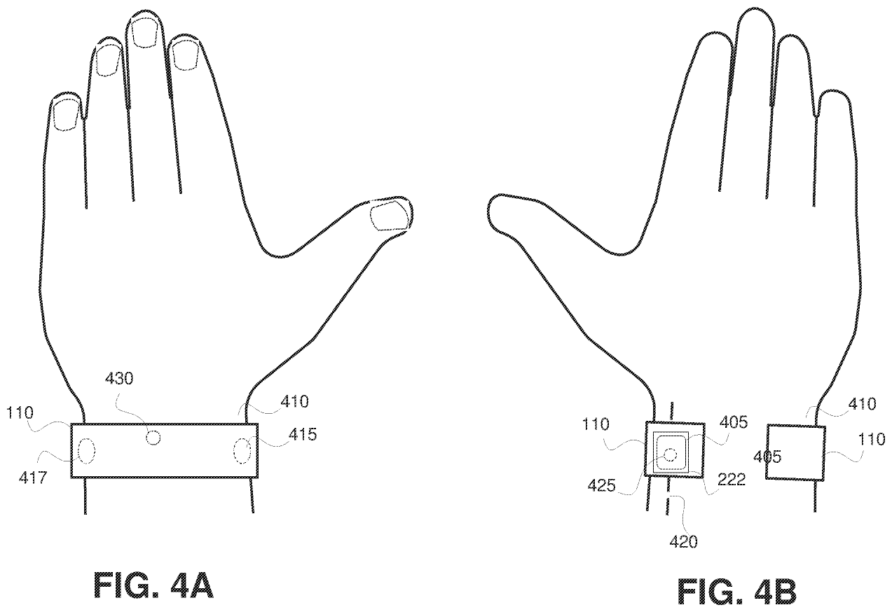

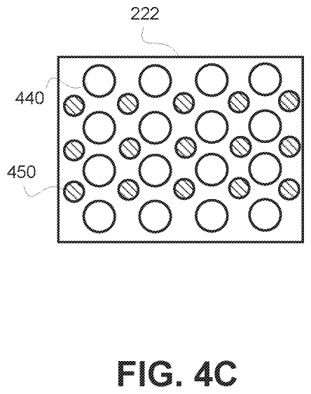

FIG. 4A and FIG. 4B are schematic diagrams illustrating an example wearable device 110. In the examples of FIG. 4A and FIG. 4B, the wearable device 110 can be carried out in a shape of an open bangle. The FIG. 4A shows a top view of a patient's hand 410 and the wearable device 110 placed on the patient wrist. FIG. 4B is an inside view of the patient's hand 410 and wearable device 110. The wearable device 110 can be designed to apply pressure at an area 405 of skin surface covering a radial artery 420. In comparison to wristbands and straps, an open bangle may be more comfortable to wear by a patient since no pressure is applied to the middle area inside the wrist. It should be noted that sensors 120 can be arranged around the wearable device to take appropriate measurements from the inside and top of the wrist of the patient.

The wearable device 110 can include optical sensors 222 located on an inner side of the wearable device 110. When the wearable device 110 is worn on the patient's hand, the inner side can be in permanent contact with a surface of the skin of the patient hand 410. The wearable device 110 can be placed around a wrist of patient's hand 410 in such a way that sensors 222 are located as close as possible to cover the skin area 405 covering the radial artery 420. The sensors 222 can be configured to be in a permanent contact with the skin of the patient 110. The radial artery is located right beneath the skin, thereby allowing measurements of oxygen saturation, heart rate, cardiac output, and other parameters by optical sensors 222 using pulse oximetry methods.

Oxygen saturation is the relative proportion (typically expressed as percentage) of oxygen dissolved in blood, as bound to hemoglobin, relative to non-oxygen-bound hemoglobin. Oxygen saturation is important in clinical monitoring of surgical anesthesia, and in monitoring and assessment of various clinical conditions such as the COPD and asthma. In healthy individuals, oxygen saturation is over 95%. Direct measurement can be made from arterial blood sample, but drawing blood is an invasive procedure, and, except for a few controlled environments (e.g. during a surgery) cannot be easily performed continuously. Pulse oximetry can yield a quantity called SpO2 (saturation of peripheral oxygen), an accepted estimate of arterial oxygen saturation, derived from optical characteristics of blood using transmission of light through a thin part of a human body, for example, a fingertip or an earlobe (in the most common transmissive application mode). Reflectance pulse oximetry can be used to estimate SpO2 using other body sites. The reflectance pulse oximetry does not require a thin section of the person's body and is therefore can be suited to more universal application such as the feet, forehead and chest, but it has some serious issues due to the light reflected from non-pulsating tissues.

In other embodiments, as shown in FIG. 4C, the optical sensors 222 include multiple light sensors 440 (photoelectric cells), to measure the reflected light, and multiple light transmitters 450 (for example, Light Emission Diodes (LEDs)). The number and location of the light sensors and light transmitters can be chosen such that in case of an accidental displacement of the wearable device, at least one of the light sensors is still located sufficiently close to the radial artery. In some embodiments, when measuring the light reflected from the skin and radial artery, a signal from those photoelectric cells that provides the strongest or otherwise determined best output can be selected for further processing in order to obtain medical parameters using methods of pulse (reflectance) oximetry. In certain embodiments, the wearable device 110 is configured to apply a pre-determined amount of pressure to the wrist each time the user wears the wearable device to allow the same conditions for the reflection of the light from the skin.

In other embodiments, when oxygen saturation cannot be measured directly from arterial blood, an indirect measurement can be performed by tracking tissue oxygen saturation. The measurement of oxygen saturation is commonly used to track progression of heart diseases or lung disease. When heart or lungs are not functioning properly, the saturation of oxygen drops in both arterial blood and tissue around the artery. Therefore, tissue oxygen saturation can be measured by sensing the skin color near the radial artery. For example, if the wearable device 110 moves so that the optical sensors 222 are not covering the radial artery, measurements of tissue saturation near the radial artery can be used as a backup to provide values for oxygen saturation. In certain embodiments, the oxygen saturation and tissue saturation can be measured simultaneously. In some embodiments, the oxygen saturation and tissue saturation can be measured using the same optical sensor.

Referring now to FIG. 4A, the wearable device 110 may include input plates 415 and 420 of electrical sensors 224. The input plates 415 and 420 can be located on the inner side of the wearable device 110. When the wearable device 110 is placed on a wrist of the patient, the input plates 415 and 420 can be positioned to be in permanent contact with the skin of the wrist of the patient. In some embodiments, the input plates (electrodes) 415 and 420 shown can be configured to be located at the opposite edges of the wrist of patient.

In some embodiments, a combination of ECG and pulse oximetry can be used to determine cardiac output. Cardiac Output (CO, Q, or Qc) is a volume of blood pumped by the ventricles of the heart per unit time, typically expressed as milliliters per minute (ml/min). The cardiac output can be directly derived from other cardiac parameters, namely as the product of stroke volume (SV, the blood volume output of the heart with each beat), and the heart rate (HR), that is, CO 32 SV*HR. Clinically, the cardiac output is an indicator of the sufficiency of blood supply. In healthy individuals at rest, CO is about 5 or 6 liters of blood per minute. During strenuous activity, CO can increase to levels more than five times the resting level. In conditions such as hypertension, valvular heart disease, congenital heart disease, arrhythmias, CO is typically reduced.

In some embodiments, a combination of ECG and pulse oximetry can be used to estimate CO directly using the equation CO=SV*HR, by least squares regression modeling of stroke volume (based either on individual direct calibration to a specific patient, or calibration to physical and clinical patient characteristics), and replacing SV by the appropriate regression expression. Specifically, pulse wave transit time, the interval between the ECG R wave peak and the pulse oximeter pulse wave foot, transformed by an appropriate regression expression, replaces SV. The CO estimate can be determined using individual heartbeat raw ECG and pulse oximetry waveform parameters, or may be a time-averaged estimate, derived from synchronized reconstructed one-handed ECG and averaged pulse oximeter readings over a specified time period. Simple changes in CO, useful in tracking individualized patient trends, can be obtained by similar means, without the necessity for absolute calibration.

In some embodiments, the wearable device 110 is operable to determine a pulse rate. The pulse rate is an indicator of a heart rate, as determined at a peripheral body site (arteries of a wrist, arm, leg, or neck). Considered as one of the vital signs, the pulse rate can be an indicator of a general health and physiological state. The pulse rate can be derived directly from any pulse-oximeter. Normal resting values can vary widely, but typically, remain within 60-100 pulsations per minute. Fluctuations in the heart rate (Heart rate variability or HRV) are normal, with higher degrees generally associated with better heart reactivity and health.

In some embodiments, the wearable device 110 is operable to determine a blood pressure (BP). The BP, another vital sign, generally refers to the intra-arterial pressure of blood at two specific stages of the heartbeat, the maximum pressure at systole (ventricular contraction) and the minimum pressure at diastole (relaxation and filling of ventricles), expressed as a delimited pair of numbers for systolic and diastolic BP respectively, in mmHg, e.g. 150/80 mmHg. The BP can be measured continuously by an invasive arterial catheter, non-invasive measurement at the arm by a stethoscope and a sphygmomanometer, or an automated cuff. A healthy adult resting BP can vary around 120/80 mmHg. High or low BPs are associated with many disease states, with long-term changes being associated with changes in the health status. Extreme short-term changes can be associated with acute disease episodes, particularly in chronically ill patients. A risk of developing a number of diseases, such as cardiovascular disease, can be associated with extreme BPs. Short-term changes in the BP can be associated with changes in physical or mental state.

According to some embodiments of present disclosure, a combination of ECG and pulse oximetry can be used to estimate systolic BP changes. The systolic BP changes can be estimated using a pulse wave transit time, the interval between the ECG R wave peak and the pulse oximeter pulse wave foot.

In certain embodiments, with a suitable calibration and individualized adjustment based on various patient characteristics, absolute estimates of the BP can be determined. The BP changes or absolute estimates can be determined using individual heartbeat raw ECG and pulse oximetry waveform parameters, or may be a time-averaged estimate, derived from synchronized reconstructed one-handed ECG and averaged pulse oximeter readings over some specified time period.

In some embodiments, the wearable device 110 is operable to determine vascular resistance. Vascular resistance is the force which opposes the flow of blood through the circulation. Typically, the systemic vascular resistance (SVR), which is the resistance of the peripheral circulation, is considered. Measurements can be expressed in several different unit systems; clinically the units are often mmHg/L/min, as SVR is a function of both blood pressure and cardiac output, that is, SVR=BP/CO. Normal values are typically within 10-20 mmHg/L/min. SVR can change as a result of various physiological stresses on the body, such as with exercise where the vascular resistance decreases, resulting in increased blood flow, or with drug or disease-related challenges.

Using measurements of ECG and pulse oximetry, the SVR can be derived as either a change or tracking score, or an absolute estimate, based on instantaneous (single heartbeat) or average BP and CO estimates.

In some embodiments, the wearable device 110 is operable to determine respiratory rate using a pulse oximetry and ECG. The respiratory rate, which is another vital sign, is typically expressed as the number of breaths per minute. Typical adult resting respiratory rate is about 16-20 breaths per minute. Extreme variations can result from physical or psychological stress. The respiratory rate is often affected in chronic disease states, particularly in pulmonary and cardiovascular disease. Extreme short-term changes may be associated with acute disease episodes, particularly in chronically ill patients.

In some embodiments, the wearable device 110 may include a temperature sensor 425 and a temperature sensor 430. The temperature sensor 425 can be located on the inner side of the wearable device 110. When the wearable device 100 is worn on patient's hand, the temperature sensor 425 can be in permanent contact with the skin of the patient. The temperature sensor 425 can be used to measure temperature of the skin (a skin temperature) of the patient at the hand. In certain embodiments, the temperature sensor 425 can be located sufficiently close to cover the skin area 405 over the radial artery 420. Because the radial artery 420 carries blood flowing from the core of the body of the patient, the temperature provided by the temperature sensor 425 is close to the core temperature of the patient. In some embodiments, the wearable device 110 may further include a temperature sensor 430. The temperature sensor 430 can be located at the outer side of the wearable device 110. The outer side is not in contact with the surface of the skin of the patient's hand. Therefore, the temperature sensor 430 can be used to measure a temperature of an external environment (an external temperature), for example, a temperature of air in a room.

In some embodiments, the wearable device 110 can be configured to estimate a body temperature of the patient based on measurements of the skin temperature and the external temperature. In some embodiments, the body temperature can be defined as a linear combination of the skin temperature and the external temperature. Coefficients of the linear combination can be determined by a calibration process using regular body temperature measurements. The calibration can be carried out at the first use of the wearable device 110 and periodically repeated during further uses of the wearable device. In other embodiments, the body temperature can be defined as a non-linear function of the skin temperature and the external temperature. Type and parameters of the non-linear function can be determined via the calibration process using regular body temperature measurements.

In further embodiments, the mobile device 110 can be operable to track levels of one or more medicine in the blood of the patient 130 for a desired period of time. The level of medicine can be analyzed in combination with other blood parameters to see trends in progression or regression of chronical diseases. Based on the trends, the patient can be provided with advice to modify times for taking the medicine and/or amounts of the medicine. The patient can be warned if the level of the medicine in the blood is too high or too low. The doctor 170 view reports on the medicine levels to ensure that the medication level is within a proper range for providing effective treatment of the chronic diseases.

In some embodiments, the wearable device 110 can facilitate monitoring trends of medical parameters of the patient during a treatment. The information concerning the trends can be further used to predict a reaction of the patient to various medicines.

In some embodiments, an analysis of trends of the medical parameters can be used to predict susceptibility of the patient to local environmental condition. For example, based on a weather forecast, a reaction of patient to a weather condition, pollen count, air pollution indices can be predicted. The patient can be given an advice, for example, to take a medicine in order to avoid worsening the health conditions.

In some embodiments, changes in monitored medical parameters can be correlated to certain social events, like news, or other external stimuli. The correlation can be used for determining psychological physiological characteristics of the patient.

In some embodiments, the monitoring of medical parameters can be combined with monitoring particular habits of the patient. The habits can be determined based on movement activity. For example, the following can be monitored: a number of steps during a day, times of waking up and going to sleep, daily time periods of performing physical exercises, and so forth. Based on the changes in medical parameters, the user can be advised, for example to change quantity and/or quality of certain activities.

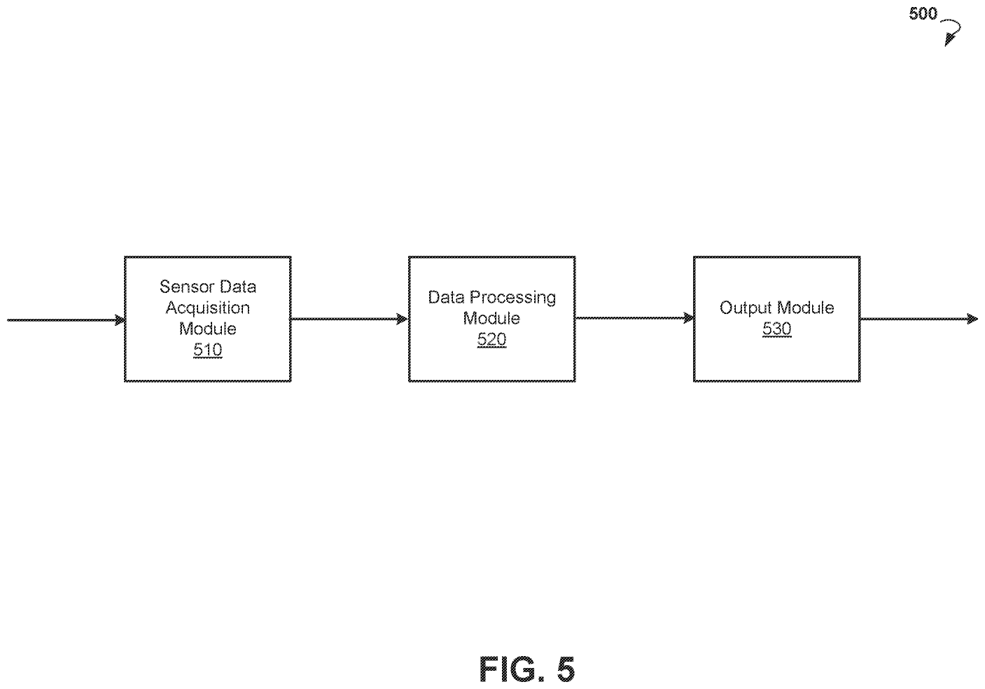

FIG. 5 is a block diagram showing components of system 500 for providing an EWS of a health status of a patient, according to an example embodiment. The system 500 can include a sensor data acquisition module 510, a data processing module 520, and output module 530. In some embodiments, the modules 510-530 are implemented as chipsets included in the wearable device 110. In other embodiments, the modules 520 and 530 can be stored as instructions in memory of the wearable device 110, mobile device 140, or computing cloud 150, and executed by a processor.

In some embodiments, the sensor acquisition module 510 is configured to receive and digitalize the sensor data. The sensor acquisition module 510 can include one or more analog-to-digital converters to transform the electrical signals from sensors to digits.

In some embodiments, the output module 530 can be configured to provide reports and alert messages concerning a health status of the patient 130.

In some embodiments, the data processing module 520 can be configured to analyze the sensor data to obtain medical parameters 310. The data processing module 520 can be configured to determine trends in the medical parameters 310 to track the health status of the patient 130. In certain embodiments, the data processing module 520 may be configured to determine a general score representing the health status of the patient. The general score can be a sum of individual scores of medical parameters being monitored while the wearable device 110 is in use. In some embodiments, the number of monitored medical parameters can be extended based on a current patient health status and environment conditions. In certain embodiments, the medical parameters can be assigned weights based on the current patient health status and environmental conditions. The weights can be used in a summation of the individual scores of the medical parameters.

In some embodiments, the monitored medical parameters may include at least a respiratory rate, an oxygen saturation, a temperature, a systolic blood pressure, a pulse rate, and a level of consciousness. In some embodiments, in order to determine the level of consciousness, the wearable device may be configured to provide a signal to the patient 130 by the alarm unit 250. The patient may be instructed to touch the wearable device 110 with the other hand. In some embodiments, the wearable device may include a touch sensor configured to sense whether the patient touched the wearable device. The level of consciousness can be determined based on whether the patient touched the wearable device after the wearable device provided a signal by the alarm unit 250.

In some embodiments, the data processing module 530 is configured to determine individual scores of the medical parameters. In certain embodiments, an individual score of a medical parameter is determined based on a deviation of a current value of the medical parameter from a normal value of the medical parameter. In some embodiments, the normal value of the medical parameter is unique to the patient 130. The normal value can be determined based on historical values of the medical parameter collected during an initial time interval, for example, several days or weeks.

In some embodiments, a range of possible values of the medical parameter is divided into intervals. Each of the intervals can be assigned a score. As a result, a scale or a table for determining an individual score of a patient, can be generated. The scores associated with the interval is zero if the interval is near the normal values of the medical parameters. The score of the interval may grow gradually up to a maximum value (for example, 3) with a deviation from the normal value on both sides of the normal value.

In some embodiments, the number of intervals and scores assigned to the intervals can be individual for the patient. In certain embodiments, the number of intervals and scores assigned to the intervals can be based on an age, a gender, a gene expression, and an ethnicity of the patient. In some embodiments, the number of intervals and scores assigned to the intervals can be based on a living environment of the patient. Thus, the scale of scores can be unique and individual to a given patient. Using a unique patient scale of scores may provide for a more precise evaluation of the general score as compared to scores generated based on normative values applicable to the population at large.

In some embodiments, the general score can be used to estimate a health status of patient. If the general score exceeds a pre-determined threshold, the patient can be issued a warning signal using, for example an alarm unit of the wearable device. In some embodiments, the warning signal can be also issued if one of the individual scores reaches a maximum value. The general score and the individual scores can be used for determining an appropriate treatment for the patient. In some embodiments, the general score can be used to determine or adjust a frequency for acquiring medical parameters and calculating individual scores.

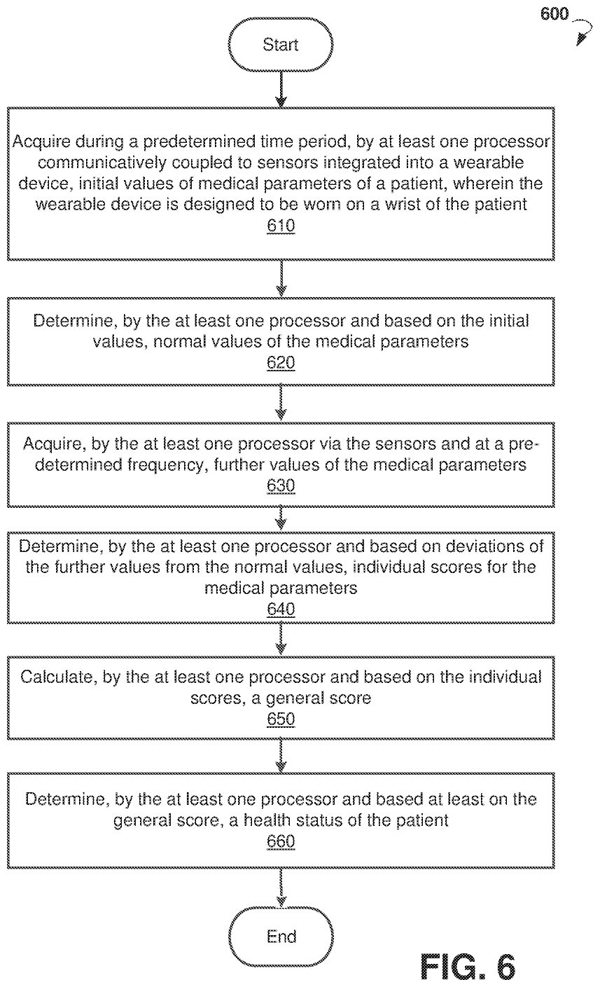

FIG. 6 is a flow chart diagram showing example method 600 for early warning of health status of patient, according to an example embodiment.

In block 610, the method 600 incudes acquiring, during a predetermined time period, by at least one processor communicatively coupled to sensors integrated into a wearable device, initial values of medical parameters of a patient. The wearable device can be designed to be worn on a wrist of the patient.

In block 620, the method 600 includes determining, by the at least one processor and based on the initial values, normal values of the medical parameters.

In block 630, the method 600 includes acquiring, by the at least one processor via the sensors and at a pre-determined frequency, further values of the medical parameters.

In block 640, the method 600 includes determining, by the at least one processor and based on deviations of the further values from the normal values, individual scores for the medical parameters.

In block 650, the method 600 includes calculating, by the at least one processor and based on the individual scores, a general score.

In block 660, the method 600 includes determining, by the at least one processor and based at least on the general score, the health status of the patient.

The present technology is described above with reference to example embodiments. Therefore, other variations upon the example embodiments are intended to be covered by the present disclosure.

### Estimation of Physiological Parameter Using Deep Neural Networks and Parameter Model

Indirect physiological measurements can be used to estimate various user's physiological status which can be verified by a direct measurement. For example, measuring an electrical signal from the heart along with an optical reading of the pulse along with other measurements and calculations can be used to estimate a person's blood pressure. This estimate can be verified by a direct measurement, a blood pressure cuff for example. The difference between the indirect physiological parameter estimate and a direct physiological measurement can be stored in a user service and database. Some service have associated wearable devices for thousands or millions of users where their associate indirect, derived, direct physiological measurements, and other user attributes are stored. These may be associated with a wearable user device that is registered with a service. The wearable user device can be in communication with the service and thus physiological measurements are updated and loaded to and stored by the service.

Each user account/database t may contain other information, including but not limited to, a user's physical and medical status that could be used by a DNN (Deep Neural Network) to determine calibration coefficients for one or more of the parameters measured by the wearable device. Further, in some embodiments this user account/database information can be used by the DNN to determine a group for each user. Alternatively, the derived parameter based model from the DNN output can be used to determine groups. A group is a cluster of users where the same calibration coefficients may provide the same range of error between the indirect estimate and the direct measurement of a physiological parameter. For example, gender, age, having high blood pressure, taking certain medications, weight and height, body mass index, or medical conditions like diabetes, heart disease, maybe included in a service's user profile. Providing accurate estimates of physiological parameter is important in accurately estimating a person's EWS.

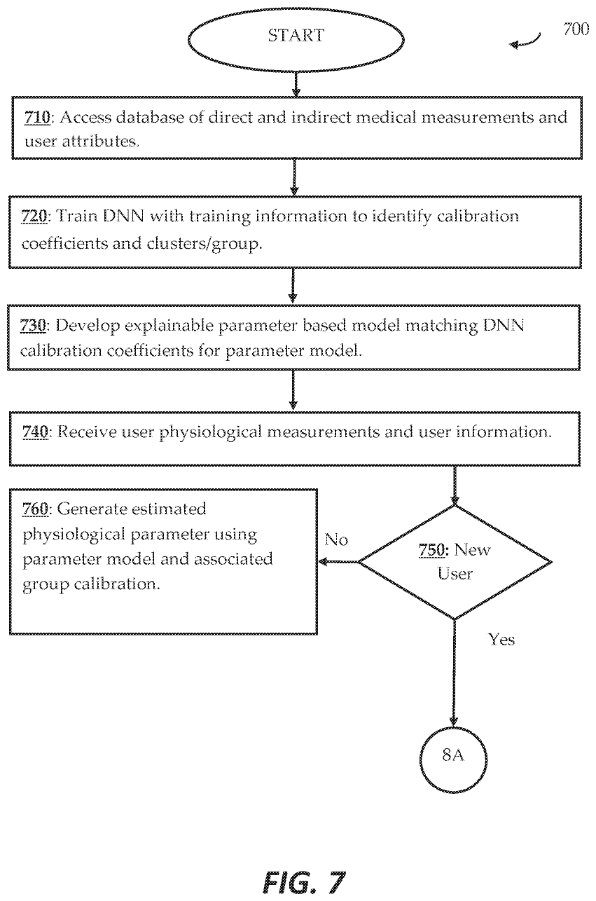

FIG. 7 shows a flow chart diagram showing example method 700 for training a DNN to estimate a physiological parameter from user data in a wearable device service database. Further from the DNN a parameter model can be determined to provide an explainable model for determining a physiological parameter. For each user, a group is assigned where determined and associated calibration coefficients provide an estimated parameter value within a error range.

For new users where their calibrated estimates do not fall within previously determined groups, new calibration coefficients and a new group needs to be defined. For example, a new user may appear to fall within a group using an associated a group calibration for estimating blood pressure. However, upon making a direct measurement, the user's direct parameter may have an error outside the range expected for the group. New calibration coefficients and a new group is needed. In step 710, a database or service containing user information is accessed. This information can include direct, indirect, and calculated physiological measurements. In one embodiment these measurements are generated by a wearable device and can include, but are not limited to, blood oxygen level, pulse rate, temperature, and pulse width velocity. The indirect physiological measurement can be used to derive or calculate an estimate of the direct physiological measurement, for example blood pressure. The database can include a history of these parameter measurements. Direct physiological measurements can also be stored in the database such as a cuff measurement of the blood pressure which is more accurate. Further, the database can include user entered information relating to a user's physical and medical status. These are factors that can be utilized by the DNN or by other calculations in determining groups of users the have the same error profile of indirect or derived physiological measurements. Accordingly, a user within the same group will use the same calibration coefficient.

In one aspect of the invention, the physiological estimate is blood pressure utilizing physiological parameter measurements, also known as parameter measurements, from a wearable device. The database can contain a calculated estimated physiological measurement based on one or more indirect physiological measurements. These indirect physiological measurements can include, but are not limited to, heart electrical signals, heart rate, pulse readings, pulse width velocity, oxygen saturation, and body and environment temperature. The DNN will use the indirect physiological measurements as inputs for training and the error between a direct physiological measurement and the indirect or calculated physiological measurement for the target output.

In step 720, the DNN is trained using data stored in the user database or other storage. The difference between the direct physiological and indirect or derived physiological measurement can be used as the training error. The output of the DNN can be an estimate corrected physiological measurement like blood pressure. In one aspect of the invention, the DNN uses user attributes including but not limited to physiological parameters and medical information to determine groups. A group is users that would have the same calibration coefficients and having similar parameter estimation errors when utilizing indirect measurements.

The user training data should be filtered before training the DNN. The filtering is to prevent biases in the data that can be causes by missing or duplicate data, different time ranges of data. A person experienced with training DNN would understand how the data should be preprocess before training a DNN.

In step 730 a parameter model is developed from the DNN. A parameter model is much simpler equation than a DNN and requires less resources to run. Of more importance is that the parameter model is explainable when there is a large error between an estimated parameter and direct parameter. A person of ordinary experience in data science and DNN would know how to use the trained DNN to determine the weights for a parameter model. For example, the parameter model equation for blood pressure may have as inputs, heart electrical signals, heart rate, pulse readings, pulse width velocity, oxygen saturation, and body and environment temperature, each scaled by a weight. The calibration coefficient for a group, in one aspect of the invention, can be a scaling factor applied directly to the estimated blood pressure or can be one or more scaling factors applied to parameter model inputs. In another aspect of the invention, the calibration coefficient can include an additive component applied to either the input parameters or the estimated output. Further, the calibration coefficient can be one or more curves based on one or more of the parameters.

In step 740, the system is in operation where it receives user physiological parameter data. This data can be from a user already in the database or from a new user. This data will be used to generate a derived physiological parameter value that can include blood pressure and a group to which the user is associated.

In step 750, it is determined if the user data is from a new user or a user that belongs to a previously determined group. If the user data belongs to a previously determined group, then the data is processed normally in step 760. If the data is for a new user, then a determination is made regarding what group the new user belongs or if a new group needs to be created with new calibration coefficients.

In step 760 the parameter correction coefficients associated with a group are selected. A estimated physiological parameter value is generated by the parameter model using the associated group calibration. The results can be stored back into the database.

In step 770 the correction coefficients for either the indirect or determined applied to one or more of the user data parameters or the output of the DNN thereby generating a corrected physiological measurement.

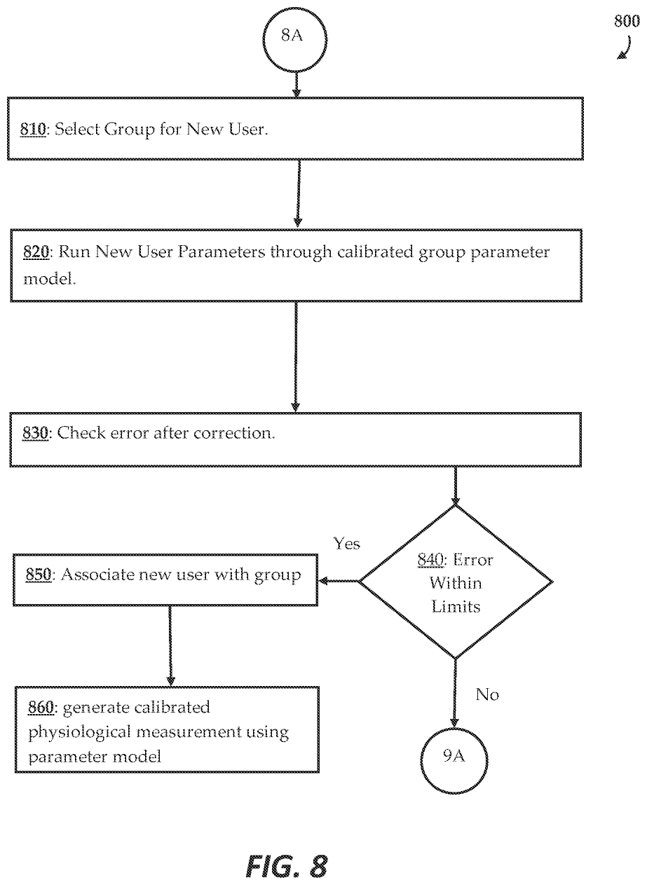

FIG. 8 shows the steps 800 for processing a new user. The steps determine if the new user fit within a predetermined group or if a new group or subgroup needs to be created. The determination of the group can be performed by a DNN or using the physiological parameter data. In one aspect of the invention, the selection of the group may include non-measured user information including but not limited to medical history and user status. In another aspect of the invention the group is determined by the physiological parameter and their distance from a mean value for a group. For example, it may be determined using a user's profile that users over the age of 50 have a grouping and utilize the same correction coefficients. The distance may be based on distance from a physiological parameter average.

In step 810, a group is selected which minimizes the error or distance for the new user. The error or distance based may be based on the new user's profile information, indirect, direct, or derived parameter measurements.

In step 820 the physiological parameter data from the new user is processed by the parameter model and group correction to determine an estimated parameter value. An exemplar parameter value is blood pressure.

In step 830, the estimated parameter value is compared against a direct measurement of the parameter value. The difference is the error value or distance.

In step 840, the error value or distance is evaluated to determine if it falls within error for the group which the new user was initially linked.

In step 850, if the error or distance is within a predetermined range, then the new user is associated with the preselected group and the calibration coefficients associated with that group can be used in processing physiological parameter data from the new user.

In step 860 the new user's physiological measurements are processed the same way as other users in the database. The parameter model and the associated group's calibration coefficients are used to generate an estimated value, for example blood pressure.

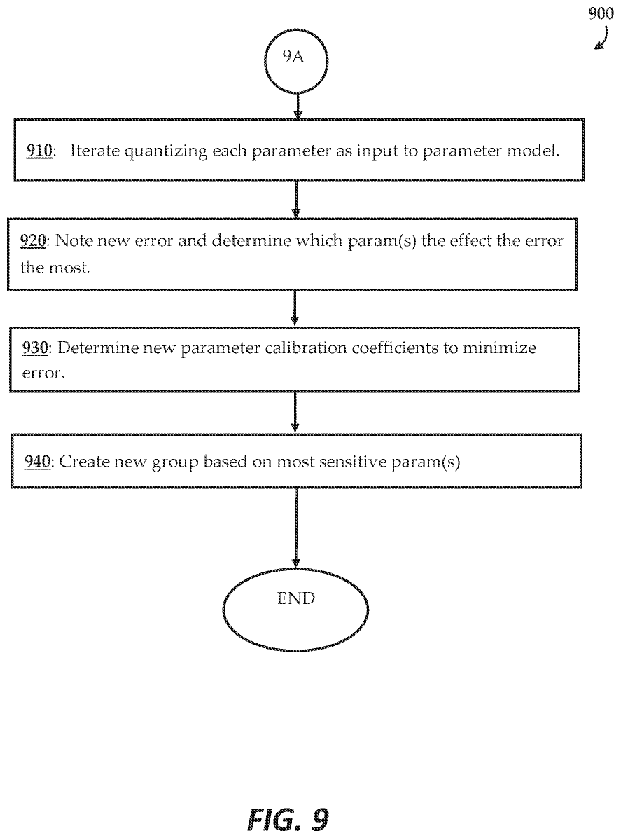

Referring to FIG. 9, the process 900 for reducing the error or distance from a group is disclosed and described. The objective is to determine which of the inputs to the parameter model most affects the error. The other technique will zero out one or more inputs and see the resulting effect. However, the is unlikely to work where some of the physiological values need to be present to determine the estimated value.

In step 910, the inputs to the parameter model are quantized. The inputs are the physiological measurements for the new user. For example, these can include but are not limited to heart electrical signals, heart rate, pulse readings, pulse width velocity, oxygen saturation, and body and environment temperature, each scaled by a weight. These can be received from a wearable device. Each input has a range of values. The quantization of the input can be done in several ways.

In one aspect of the invention the quantization is provided by using values a specified percentage greater and lesser than the new user physiological measurement input. For example, this may include quantized values 10% and 20% greater and 10% and 20% less than the new user value for a parameter. The number of quantization levels can increase or decrease, and the percentage change can also be greater or smaller. For example, if the physiological value has a range of one to ten and the value six is received, then the parameter model will be run using 4.8, 5.4, 6.6, and 7.2.

In another aspect of the invention the quantization is performed by adding and subtracting a fixed increment to the new user physiological parameters received.

For example, the increment may be 0.5. If the physiological parameter received is 6, the parameter model is executed with the values 5.0, 5.5, 6.5 and 7.0.

In step 920, the parameter model is evaluated with the quantized parameter values utilizing the correction associated with the group closest to the new user. The one or more quantized physiological parameter that generates the smallest error is noted.

In step 930, new parameter calibration coefficient to minimize the error are determined for the new user.

In step 940, based on the new user's physiological parameters, a new group is formed with an associated correction. The new user can be stored in the user database.

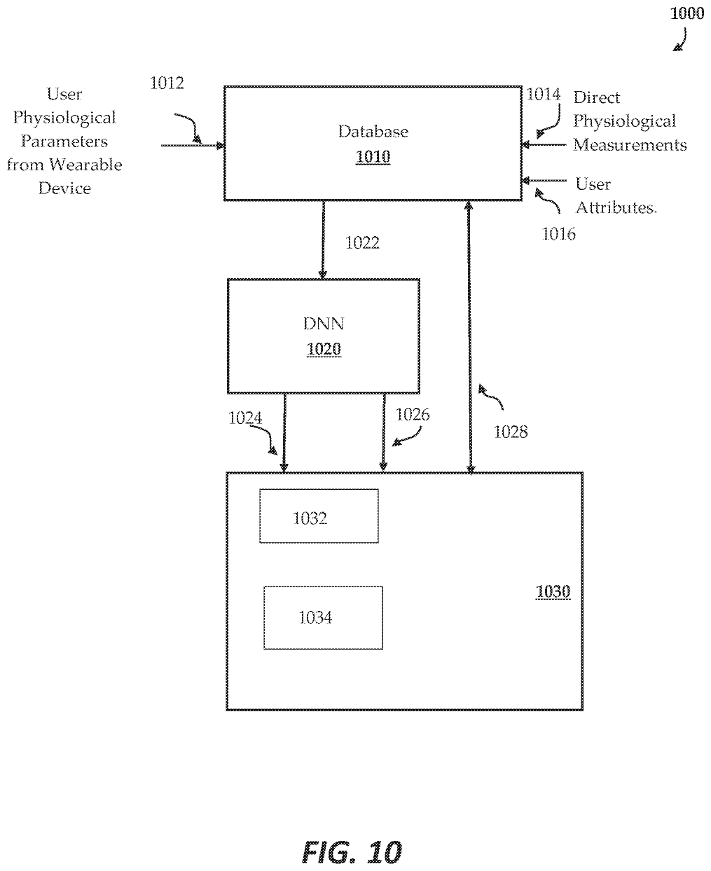

Referring to FIG. 10, a block diagram showing one embodiment of a system 1000 configured to estimate physiological parameters utilizing a deep neural network to build calibrated parameter model is shown and described below.

The system 1000 can include a wearable device (FIG. 1-110) that communicates physiological parameters 1012 measured from a user (FIG. 1-130) to a user database 1010. Exemplar of the physiological parameters 1012 include but are not limited to respiratory rate, an oxygen saturation, a temperature, a derived blood pressure, and a pulse rate. The database 1010 can include direct physiological parameter measurements. Exemplar of a direct physiological parameter is blood pressure taken by and blood pressure cuff with a doctor using a stethoscope to detect the systolic and diastolic blood pressure. Further, a user may configure an account in the database 1010 with additional physical and medical information 1016 that can be useful in determining groups and for the DNN to improve the accuracy of physiological parameter estimates. These can include gender, age, blood pressure, medications being taken, weight and height, body mass index, or medical conditions like diabetes, heart disease, and food and drinking preferences.

The system includes computational components for performing the DNN 1020. This hardware can include a general-purpose processor or server and software. Further, the DNN 1020 can include special purpose neural network processors for processing acceleration.

The DNN 1020 can receive physiological parameter measurements data 1012, 1014 and user attribute data 1016 from the database 1010. The DNN 1020 outputs physiological estimates 1024 and can output groups 1026. These outputs 1024, 1026 are received by the processing component 1030.

The processing component 1030 can compute the calibration coefficients and/or curves for the physiological parameters. Alternatives, the calibration coefficients and/or curves can be received from the database for use with user physiological parameter data. The component 1032 can generate new coefficients for new user groups or provide the calibration for the parameter model 1034. The processing component 1030 includes a processing component that determines a parameter model 1034. The processing component 1030 is responsible for implementing accessing the database 1010 for receiving user physiological parameters 1028, group information 1028 for a user and uploading new users group information and calibration information for a new user 1028.

What is claimed is:

1. A method for estimating a physiological parameter using a parameter model determined by a deep neural network, the method comprising:
accessing a user database containing one or more indirect and direct physiological parameters for each of a plurality of users by at least one processor;
training a DNN (deep neural network) with the plurality of users one or more indirect physiological parameters and where the direct physiological parameter is a training target;
determining one or more groups from the users physiological parameters;
associating each user in the user database with one or more groups;
determine a calibration for each of the one or more groups;
generating a parameter model that substantially matches the deep neural network;
receiving a new user physiological parameters;
determining a group distance of the new user from each of the one or more groups;
determining the closest group;
determining the error of the parameter model using the closest group and associated calibration;
if the error is greater than a threshold,
quantizing the new user physiological parameters;
iterate one at a time each new user physiological parameters input to the parameter model;
determining the one or more quantized new user physiological parameters that reduces error; and
create new group based on the new user physiological parameters.

2. The method of claim 1, wherein the one or more groups are determined by the euclidian distance of a user physiological parameters from a group's physiological parameters.

3. The method of claim 1, wherein the one or more groups are determined by utilizing a DNN.

4. The method of claim 1, wherein the calibration for a group utilizes scalar coefficients applied to the physiological parameters of a user in a group.

5. The method of claim 1, wherein the calibration for a group utilizes scalar coefficients applied to the output of the parameter model.

6. The method of claim 1, wherein the one or more indirect physiological parameters are one or more of respiratory rate, an oxygen saturation, a temperature, a derived blood pressure, and a pulse rate.

7. The method of claim 6, wherein the indirect physiological parameter is a directly measured blood pressure.

8. The method of claim 1, wherein the quantizing the new user physiological parameters selects four parameter levels for each new user physiological parameter, the quantized parameter levels being twenty and ten percent below the new user physiological parameter and ten and twenty percent above the new user physiological parameter.

9. The method of claim 1, wherein the quantizing the new user physiological parameters selects four parameter levels equally spaced between minimum and maximum values of the physiological

10. The method of claim 1, wherein the parameter model is used for determining an early warning score for a health status of a user.

11. A system for estimating a physiological parameter using a parameter model determined by a deep neural network, the system comprising:
at least one processor communicatively coupled to a user database and a wearable device; and
a memory communicatively coupled with the at least one processor, the memory storing instructions, which when executed by the at least one processor performs a method comprising:
accessing a user database containing one or more indirect and direct physiological parameters for each of a plurality of users by at least one processor;
training a DNN (deep neural network) with the plurality of users one or more indirect physiological parameters and where the direct physiological parameter is a training target;
determining one or more groups from the users physiological parameters;
associating each user in the user database with one or more groups;
determine a calibration for each of the one or more groups;
generating a parameter model that substantially matches the deep neural network;
receiving a new user physiological parameters;
determining a group distance of the new user from each of the one or more groups;
determining the closest group;
determining the error of the parameter model using the closest group and associated calibration;
if the error is greater than a threshold, quantizing the new user physiological parameters;
iterate one at a time each new user physiological parameters input to the parameter model;
determining the one or more quantized new user physiological parameters that reduces error; and
create new group based on the new user physiological parameters.

12. The system of claim 10, wherein the one or more groups are determined by the euclidian distance of a user physiological parameters from a group's physiological parameters.

13. The system of claim 10, wherein the one or more groups are determined by utilizing a DNN.

14. The system of claim 10, wherein the calibration for a group utilizes scalar coefficients applied to the physiological parameters of a user in a group.

15. The system of claim 10, wherein the calibration for a group utilizes scalar coefficients applied to the output of the parameter model.

16. The system of claim 10, wherein the one or more indirect physiological parameters are one or more of respiratory rate, an oxygen saturation, a temperature, a derived blood pressure, and a pulse rate.

17. The system of claim 16, wherein the indirect physiological parameter is a directly measured blood pressure.

18. The system of claim 10, wherein the quantizing the new user physiological parameters selects four parameter levels for each new user physiological parameter, the quantized parameter levels being twenty and ten percent below the new user physiological parameter and ten and twenty percent above the new user physiological parameter.

19. The system of claim 10, wherein the quantizing the new user physiological parameters selects four parameter levels equally spaced between minimum and maximum values of the physiological

20. The system of claim 10, wherein the parameter model is used for determining an early warning score for a health status of a user.
